(19) United States
(12) Patent Application Publication
(10) Pub. No.: US 2025/0034502 A1
(43) Pub. Date: Jan. 30, 2025

(54) AGITATOR FOR A BIOREACTOR, AGITATOR UNIT, BIOREACTOR, PRODUCTION METHOD AND METHOD FOR OPERATING A BIOREACTOR

(71) Applicant: EPPENDORF SE, Hamburg (DE)

(72) Inventor: Anna Schütt, Hamburg (DE)

(73) Assignee: EPPENDORF SE, Hamburg (DE)

(21) Appl. No.: 18/716,760

(22) PCT Filed: Dec. 7, 2022
(86) PCT No.: PCT/EP2022/084739

§ 371 Date: Jun. 5, 2024

(30) Foreign Application Priority Data
Dec. 7, 2021 (EP) 21212832.6

(51) Int. Cl. C12M 1/06; C12M 1/00

(52) U.S. Cl. CPC C12M 27/06; C12M 23/38

(57) ABSTRACT

An agitator for a bioreactor includes an agitator shaft having an agitator unit with an agitator blade receiving portion and at least one agitator blade arranged on the agitator blade receiving portion. The at least one agitator blade is movable between a first angular position and a second angular position, wherein the at least one agitator blade is oriented at a first angle of attack in the first angular position and at a second angle of attack in the second angular position.

### CROSS-REFERENCE TO FOREIGN PRIORITY APPLICATION

The present application claims the benefit under 35 U.S.C. §§ 119(b), 119(e), 120, and/or 365(c) of International Application No. PCT/EP2022/084739 filed Dec. 7, 2022, which claims priority to European Application No. EP 21212832.6 filed Dec. 7, 2021.

### FIELD OF THE INVENTION

The invention concerns an agitator for a bioreactor, an agitator unit for equipping an agitator shaft, and a bioreactor. The invention also concerns a method for producing an agitator unit and/or an agitator for a bioreactor, a method for producing a bioreactor, and a method for operating a bioreactor.

### BACKGROUND OF THE INVENTION

Bioreactors, frequently also called fermenters, are container systems in which certain microorganisms, cells, or plants are cultivated, preferably under optimal, controlled and reproducible conditions, in order to obtain actual cells or parts thereof or of at least one of their metabolic products. For this, bioreactors enclose a reaction space which can receive a medium, in particular a culture medium.

Different types of bioreactors are known, such as, e.g., so-called agitation vessel reactors, also called agitation reactors. Such bioreactors have differently designed agitation functions for mixing and in particular homogenizing the substances present in the reaction space, in particular the culture medium. The mixing and in particular homogenizing takes place for example via a rotational movement of at least one agitator blade arranged on agitator shaft. For this, the agitator shaft is set in rotation in particular by a drive unit.

Various agitator types and properties are known. Selection of a suitable agitator type in particular depends on the agitation task, the viscosity of the culture medium, the shear strength, but also on what agitation power is available or necessary for the process. For example, the following main agitation tasks can be distinguished: homogenization, dispersion, heat transfer, suspension, and emulsification. These agitation tasks rarely occur in isolation, so it is necessary to perform different agitation tasks simultaneously or successively in a temporal sequence.

Usually, different agitator types are suitable or preferred for the different agitation tasks. Therefore, when selecting an agitator type, often a compromise must be made between an “optimal” agitator type for key process steps, and general suitability for other applicable method steps, which are however less relevant for the yield and quality of the product.

Usually, bioreactors are operated in a continuous process. In particular, to avoid risk of contamination, agitator units are not normally exchanged during operation. Another challenge for selection of an agitator type is the fact that often the properties of the culture medium change during a process, for example from a low-viscosity Newtonian behavior to greatly structurally viscous, non-Newtonian flow behavior. This may in particular lead to an uneven distribution of the viscosity within the bioreactor. For example, homogenization and bulk transport can thereby be negatively affected.

Also, such agitators are installed in single-use bioreactors. Here, usually a single agitator type is selected and hence only a specific agitator type can be used in the process. If a particular medium is to be processed in the bioreactor, the user must decide on an agitator type in advance. The user can no longer vary the fluid delivery during the process and in particular adapt to the changing parameters.

The invention is therefore based on the object of providing an improved solution which addresses at least one of the above-mentioned problems. In particular, it is an object of the invention to provide a solution which can provide the different agitation properties as required and at the same time allow continuous process control.

### SUMMARY OF THE INVENTION

According to a first aspect, the above-mentioned object is achieved by an agitator for a bioreactor as disclosed herein, comprising an agitator shaft having an agitator unit with an agitator blade receiving portion and at least one agitator blade which is arranged on the agitator blade receiving portion and is movable between a first angular position and a second angular position, wherein the at least one agitator blade is oriented at a first angle of attack in the first angular position and at a second angle of attack in the second angular position. The at least one agitator blade comprises a torsionally stiff rotation element which is mounted on the agitator blade receiving portion so as to be rotatable between a first rotational position and a second rotational position. The at least one agitator blade is oriented at the first angle of attack in the first rotational position and at the second angle of attack in the second rotational position. The agitator blade receiving portion comprises at least one receiving opening in which the rotation element is mounted so as to be rotatable between the first rotational position and the second rotational position. The agitator unit furthermore comprises a first stop which in the first angular position limits a movement of the at least one agitator blade in a first movement direction, and/or a second stop which in the second angular position limits a movement of the at least one agitator blade in a second movement direction, wherein preferably the first stop and/or the second stop is arranged on or formed by the agitator blade receiving portion, wherein the receiving opening forms the first stop and/or the second stop or is delimited by the first stop and/or the second stop.

The invention is based inter alia on the knowledge that different agitators, e.g., sloping blade agitators and disc agitators, are used depending on the agitation properties to be achieved. Sloping blade agitators usually comprise mostly rectangular agitator blades arranged obliquely. Also, sloping blade agitators substantially deliver axially with a radial proportion. Therefore, sloping blade agitators may be used in particular for suspension, homogenization, and/or heat exchange. Sloping blade agitators are particularly preferred in shear-sensitive processes. Disc agitators, also called Rushton turbines, usually comprise vertical agitator blades. In particular the agitation effect here is based on an outwardly directed flow with axial suction from top and bottom. The outflowing fluid is here usually subjected to high shear forces. Radial delivery may advantageously be used in particular for emulsification and gasification.

In the solution described here, an agitator is provided with an agitator shaft and agitator unit, which implements different agitator types in one agitator. Depending on a desired agitation property, a choice between two agitator types may be made, and in particular switched as required. Switching between two agitator types is possible in particular since the at least one agitator blade is arranged on the agitator blade receiving portion such that it can be moved to and fro between the first angular position and the second angular position, in order to be able to orient the at least one agitator blade at different angles of attack. Thus, for example, a sloping blade agitator and a disc agitator can be integrated in one agitator and/or agitator unit, and used as required.

Because the agitator implements two different agitator types in one agitator, the user may decide between these two agitator types during a continuous process, in particular depending on requirement. Thus the user is able to switch between these agitator types within a continuous process in order thereby to change a fluid delivery, in particular a fluid delivery direction, during this process and in particular adapt to the medium to be delivered.

Preferably, the agitator unit may be arranged on or formed on the agitator shaft.

Depending on design and size in particular, agitator units can be exchanged or mounted on the agitator shaft depending on the required agitation properties. In particular, the agitator unit may be releasably fastened to the agitator shaft. Thus the agitator unit can be exchanged depending on the required agitation properties. This design may, for example, also allow re-equipping of an agitator shaft.

The agitator blade receiving portion may, for example, be part of the agitator shaft and/or be formed by the agitator shaft itself. Alternatively, the agitator blade receiving portion may be arranged on the agitator shaft as an additional element.

An agitator shaft receiving portion may in particular mean a portion on which the at least one agitator blade is arranged. The at least one agitator blade may here either be fastened to the agitator blade receiving portion or be formed thereon. The actuator blade receiving portion may here in particular be configured such that the at least one agitator blade is arranged, in particular mounted, movably on the agitator blade receiving portion.

Preferably, two, three, or more agitator blades may be arranged on the agitator blade receiving portion. Particularly preferably, in particular six agitator blades may be arranged on the agitator blade receiving portion. Thus particularly preferred agitation properties can be achieved.

An agitator blade may here be configured in particular with right angles, in particular rectangular. Alternatively, other shapes are conceivable. For example, a shape with rounded corners is also conceivable. In particular, the at least one agitator blade extends outward starting from the agitator blade receiving portion and hence relative to a longitudinal axis of the agitator shaft.

An angle of attack of the at least one agitator blade may in this case in particular mean an angle between the at least one agitator blade, in particular a surface normal vector of the at least one agitator blade, and a virtual plane arranged orthogonally to a rotational axis of the agitator shaft. The angle of attack may here preferably be varied between more than 0° and less than 180°. Thus, all angles of attack can be achieved.

The angle of attack in particular has an influence on a flow field. The flow field can therefore be varied by changing the angle of attack of the at least one agitator blade during a process.

The first angle of attack and/or the second angle of attack may be set, in particular depending on a rotational direction of the agitator shaft, by the movable arrangement of the at least one agitator blade on the agitator blade receiving portion. Thus, in particular, by changing the rotational direction of the agitator shaft, the user may switch between two agitator types. In particular, the switch may be achieved by a force acting on the at least one agitator blade, preferably as a result of a rotation of the agitator shaft.

The agitator described in this solution is preferably configured so that the at least one agitator blade is movably arranged on the agitator blade receiving portion such that when a force acting against a first rotational direction of the agitator shaft acts on the at least one agitator blade, the at least one agitator blade is moved into the first angular position and/or fixed in the first angular position without fastening means, and/or the at least one agitator blade is movably arranged on the agitator blade receiving portion such that when a force acting against a second rotational direction of the agitator shaft acts on the at least one agitator blade, the at least one agitator blade is moved into the second angular position and/or fixed in the second angular position without fastening means.

Preferably, the agitator shaft can be driven in the first rotational direction, in particular clockwise, and alternatively in the second rotational direction, in particular counterclockwise. Rotation of the agitator shaft in the first or second rotational direction can in particular cause a force acting against this rotational direction to act on the at least one agitator blade. Thus the at least one agitator blade may be rotated into the corresponding angular position. The corresponding force may be generated in particular by the medium and/or the rotation of the agitator shaft.

In particular if the movement of the at least one agitator blade in one direction is limited, the at least one agitator blade can be fixed in one of the angular positions without fastening means.

With this embodiment, a particularly simple agitator which comprises two different agitator types can be provided.

The agitator comprises a first stop which, in the first angular position, limits a movement of the at least one agitator blade in a first movement direction, and/or a second stop which, in the second angular position, limits a movement of the at least one agitator blade in a second movement direction.

Preferably, the first movement direction may mean a movement from the second angular position towards the first angular position. Further preferably, the second movement direction may mean a movement from the first angular position towards the second angular position. Preferably, the first movement direction and the second movement direction may here be substantially opposite one another.

The first stop and/or the second stop are arranged on or formed by the agitator blade receiving portion. For example, an additional delimiting element may be provided which is arranged on the agitator blade receiving portion and forms the first stop and/or the second stop. For example, several additional delimiting elements may also be provided which form the first stop and/or the second stop and/or further stops, in particular a third stop and a fourth stop.

A stop accordingly means any possible design which may limit a movement in the first movement direction or second movement direction. For example, a surface or an edge may form the respective stop.

For example, the additional delimiting element may be provided having both a first surface or a first edge and also a second surface or second edge. The first surface or first edge may for example form the first stop. The second surface or second edge may for example form the second stop. Alternatively, the first stop and/or the second stop may be formed for example by a protrusion or web.

The rotation element may for example be formed on a side of the at least one agitator blade facing the agitator shaft, in order to connect the at least one agitator blade to the agitator blade receiving portion. The rotation element may preferably be cylindrical. Thus the rotatable mounting can be achieved particularly simply.

The receiving opening may preferably be in particular round, preferably circular, for rotatable mounting of the rotation element. Particularly preferably, this round, preferably circular receiving opening may be delimited by the first stop and/or the second stop so as to limit a movement in the first movement direction and/or the second movement direction.

Particularly preferably, a first delimiting element may be provided which is arranged on the agitator blade receiving portion and extends into the receiving opening, thereby forming the first stop and/or the second stop. In particular, it is preferred that a second delimiting element is arranged on the agitator blade receiving portion and extends into the receiving opening, thereby forming a third stop and/or a fourth stop. For example, the movement of the at least one agitator blade in the first movement direction may thus be limited by the first stop and the third stop. In particular, the movement of the at least one agitator blade in the second movement direction may be limited by the second stop and the fourth stop.

Particularly preferably, the rotation element may be fixed by form fit, in particular latched, in the receiving opening. This allows a particularly simple arrangement and fastening of the at least one agitator blade on the agitator blade receiving portion. For example, the rotation element may thus comprise a latching element which can latch in the receiving opening.

An agitator blade according to this solution may preferably have a blade region which is connected torsionally stiffly to the rotation element.

The at least one receiving opening may here be formed preferably radially and/or substantially orthogonally to a rotational axis of the agitator shaft.

According to a particularly preferred embodiment of the agitator for a bioreactor, it is preferred that this agitator comprises the agitator shaft and the agitator unit arranged on the agitator shaft, wherein the agitator unit comprises the agitator blade receiving portion, the at least one agitator blade, and the rotation element which is connected torsionally stiffly to the agitator blade and is mounted on the agitator blade receiving portion so be rotatable between the first rotational position and the second rotational position, wherein the at least one agitator blade is oriented at the first angle of attack in the first rotational position and at the second angle of attack in the second rotational position.

In operation, therefore, the agitator blade may be oriented either at the first angle of attack or at the second angle of attack.

The rotation element may here preferably be mounted rotatably on the agitator blade receiving portion such that when a force acting against a first rotational direction of the agitator shaft acts on the at least one agitator blade, the rotation element is moved into the first rotational position and/or fixed in the first rotational position without fastening means, and/or the rotation element is mounted rotatably on the agitator blade receiving portion such that when a force acting against a second rotational direction of the agitator shaft acts on the at least one agitator blade, the rotation element is moved into the second rotational position and/or fixed in the second rotational position without fastening means.

The rotation element is preferably configured to be moved from the second rotational position into the first rotational position by rotation about its longitudinal axis in the first rotational direction, and from the first rotational position to the second rotational position by rotation about its longitudinal axis in the second rotational direction.

Preferably, the first stop, against which the rotation element can stop in the first rotational position, may be arranged on the agitator blade receiving portion. Particularly preferably, the second stop, against which the rotation element can stop in the second rotational position, may be arranged on the agitator blade receiving portion.

Here preferably, the rotation element may bear against the first stop in the first rotational position and against the second stop in the second rotational position.

Preferably, the rotation element may have a cross-sectional shape which forms a first counter-stop which bears against the first stop in the first rotational position. Preferably, the cross-sectional shape of the rotation element may form a second counter-stop which bears against the second stop in the second rotational position. For example, the rotation element may have a substantially round, in particular circular cross-sectional shape with at least one flattened portion, preferably two flattened portions. The respective counter-stop may preferably be formed by a flattened portion.

Particularly preferably, the cross-sectional shape of the rotation element may form a first counter-stop and/or a fourth counter-stop.

Preferably, the third counter-stop may bear against the third stop in the first rotational position. Particularly preferably, the fourth counter-stop may bear against the fourth stop in the second rotational position.

For example, in the first rotational position, the first counter-stop may also bear against the first stop and the second counter-stop against the third stop. Preferably, in the second rotational position, the first counter-stop may bear against the second stop and the second counter-stop against the fourth stop.

According to a particularly preferred embodiment, it is provided that the receiving opening forms the first stop and/or the second stop, or is delimited by the first stop and/or the second stop.

Preferably, a disc which delimits the receiving opening and forms the first stop and/or the second stop may be arranged on the agitator blade receiving portion. Particularly preferably, the disc may be arranged on an outside or an inside of the agitator blade receiving portion.

For example, the receiving opening may have an opening cross section and/or opening cross-sectional form which forms the first stop and/or the second stop. Particularly preferably, the receiving opening may in particular have a round, in particular circular opening cross-sectional form. The respective stop may be formed in particular by a flattened region of the round, in particular circular opening cross-sectional form.

According to a particularly preferred embodiment, the agitator comprises a guide device which is configured to guide a movement of the agitator blade between the first angular position and the second angular position.

Particularly preferably, the rotation element may have a guide element which is configured to guide a rotation of the rotation element between the first rotational position and the second rotational position. Preferably, the agitator blade receiving portion may have a corresponding guide means which is configured to cooperate with the guide element of the rotation element in order to guide the rotation of the rotation element between the first rotational position and the second rotational position. Preferably, the guide element and the guide means may form a guide element system.

Preferably, the guide element may be formed as a protrusion which protrudes radially from a surface or substantially axially and/or axially-radially from a free end of the rotation element. Particularly preferably, the corresponding guide means may be formed as a guide rail which is arranged or formed on the agitator blade receiving portion or formed by an edge of the receiving opening defining the inner periphery, wherein the protrusion is guided in the guide rail.

Preferably, the guide element may be configured as a latching element, in particular a latching hook which protrudes radially or axially or radially-axially from a free end of the rotation element. Preferably, the receiving opening may form a corresponding guide means in which the guide element engages and latches.

The guide means and/or the guide element may here preferably form the first stop and/or the second stop, or the first counter-stop and/or the second counter-stop.

Preferably, the corresponding guide means, in particular the guide rail, may be formed by an edge of the receiving opening formed on a side facing away from the at least one agitator blade, wherein the guide element may be formed on the free end of the rotation element and engages behind this edge in operating state.

According to a further preferred embodiment, it may be provided that the agitator blade receiving portion and the agitator shaft are formed as one piece.

In particular, it may be provided that a middle piece arranged on the agitator shaft forms the agitator blade receiving portion. Preferably, the middle piece may be fastened to the agitator shaft. Particularly preferably, the middle piece may be fastened releasably to the agitator shaft.

Preferably, the middle piece may be formed as a solid cylinder or a hollow cylinder or a ring.

Accordingly, the agitator unit can be connected to the agitator shaft in particular fixedly or releasably, and be exchangeable.

Preferably, the middle piece may have snap hooks configured to fasten the agitator blade receiving portion to the agitator shaft. Preferably, the snap hooks may extend substantially axially from an edge of the middle piece and be configured to be brought into engagement with corresponding snap means on the agitator shaft.

Particularly preferably, the middle piece is formed as a hollow cylinder with a central cylinder opening. Preferably, the central cylinder opening may be configured to arrange and/or fasten the agitator blade receiving portion on the agitator shaft.

Preferably, the middle piece may be configured as a ring with a central ring opening. Preferably, the central ring opening may be configured to arrange and/or fasten the agitator blade receiving portion on the agitator shaft.

Particularly preferably, the agitator may thus be provided as a unit or comprise components which are separable from one another, in particular the agitator shaft and agitator unit, and/or the agitator blade receiving portion and/or the agitator blade. The separable components may thus preferably be assembled and used as required.

According to a further preferred embodiment, it is provided that the first angle of attack is less than 90° or equal to 90° to a virtual plane arranged orthogonally to a rotational axis of the agitator shaft, and/or the second angle of attack is more than 90° or equal to 90° and less than 180° to the virtual plane arranged orthogonally to the rotational axis of the agitator shaft.

It is particularly preferred if the first angle of attack or the second angle of attack is substantially 90° to a virtual plane arranged orthogonally to a rotational axis of the agitator shaft, and the respective other angle of attack deviates from 90° and, e.g., is 60°. Thus, in particular, the agitator may comprise a disc agitator and a sloping blade agitator, and hence provide different agitation properties.

Embodiments in which the agitator comprises sloping blade agitators in both angular positions are also possible. For example, one of the angles of attack may be 30° and the other angle of attack 45°.

Preferably, it is provided that the first angle of attack and the second angle of attack define different angular positions.

It is further preferred that the agitator shaft and/or the agitator unit are formed at least partly from or comprise plastic, or are formed from or comprise special steel. Alternatively, further or other materials may be used which meet the requirements for use in the bioreactor. Preferably, the agitator shaft and/or the agitator unit are produced at least partially in the injection molding process or in an additive manufacturing process. For example, the agitator shaft and/or the agitator unit may be produced by 3D printing.

Preferably, the agitator shaft and/or the agitator blade receiving portion and/or the agitator blade and/or in some cases the rotation element may be formed from or comprise plastic or be formed from or comprise special steel, and/or be produced in the injection molding process or in an additive manufacturing process.

Particularly preferably, it is provided that the agitator comprises at least one disc agitator arranged on the agitator shaft axially spaced from the agitator unit.

Preferably, the agitator may comprise at least one sloping blade agitator arranged on the agitator shaft axially spaced from the agitator unit.

Particularly preferably, the agitator may comprise at least one further agitator unit arranged on the agitator shaft axially spaced from the agitator unit and having a further agitator blade receiving portion and at least one further agitator blade, which is arranged on the further agitator blade receiving portion and is movable between two different angular positions in which the at least one further agitator blade is oriented at different angles of attack.

The further agitator unit arranged on the agitator shaft may preferably in particular be configured substantially identically to the above-described agitator shaft. Preferably, the further agitator unit may implement different angles of attack, so that the at least one further agitator blade can be oriented at a third angle of attack and a fourth angle of attack. Alternatively, at least one angle of attack may be identical to the first angle of attack and/or the second angle of attack.

Particularly preferably, the further agitator unit may comprise an agitator blade with a torsionally stiff rotation element which can be mounted on the further agitator blade receiving portion so as to be rotatable between two rotational positions in which the at least one further agitator blade is oriented at different angles of attack.

Particularly preferably, the at least one further agitator unit and/or the at least one disc agitator and/or the at least one sloping blade agitator may be arranged axially spaced from the agitator unit such that the at least one further agitator unit and/or the at least one disc agitator and/or the at least one sloping blade agitator has an effect on the flow in the medium only above a defined filling level. In this way, the at least one further agitator unit and/or the at least one disc agitator and/or the at least one sloping blade agitator may be activated depending on filling level. This embodiment is advantageous in particular since a volume and hence a filling level can change in particular during a process. Often the necessary agitation properties can also change simultaneously with the change in volume and filling level.

Particularly preferably, the agitator may comprise the at least one further agitator unit, the at least one agitator blade of which is movable, in particular mounted movably, between the first angle of attack and/or the second angle of attack, and/or a third angle of attack and/or a fourth angle of attack. Preferably, the at least one agitator blade of the further agitator unit may be oriented
at the first angle of attack in the first rotational position and at the second angle of attack in the second rotational position, or
at a third angle of attack in the first rotational position and at a fourth angle of attack in the second rotational position, or
at the second angle of attack in the first rotational position and at the first angle of attack in the second rotational position, or
at the first angle of attack in the first rotational position and at a third angle of attack in the second rotational position, or
at a third angle of attack in the first rotational position and at the first angle of attack in the second rotational position, or
at the second angle of attack in the first rotational position and at a third angle of attack in the second rotational position, or
at a third angle of attack in the first rotational position and at the second angle of attack in the second rotational position.

According to a further aspect, an agitator unit is provided for equipping an agitator shaft, comprising an agitator blade receiving portion and at least one agitator blade which is arranged on the agitator blade receiving portion and is movable between a first angular position and a second angular position, wherein the at least one agitator blade is oriented at a first angle of attack in the first angular position and at a second angle of attack in the second angular position. The at least one agitator blade comprises a torsionally stiff rotation element which is mounted on the agitator blade receiving portion so as to be rotatable between a first rotational position and a second rotational position. The at least one agitator blade is oriented at the first angle of attack in the first rotational position and at the second angle of attack in the second rotational position. The agitator blade receiving portion comprises at least one receiving opening in which the rotation element is mounted so as to be rotatable between the first rotational position and the second rotational position. The agitator unit furthermore comprises a first stop which in the first angular position limits a movement of the at least one agitator blade in a first movement direction, and/or a second stop which in the second angular position limits a movement of the at least one agitator blade in a second movement direction, wherein preferably the first stop and/or the second stop are arranged on or formed by the agitator blade receiving portion, wherein the receiving opening forms the first stop and/or the second stop or is delimited by the first stop and/or the second stop.

According to a further aspect, a bioreactor is provided which comprises a header plate and a container, wherein the header plate and the container enclose a reaction space, an agitator as described herein arranged in the reaction space, wherein the agitator shaft is mounted in a bearing so as to be rotatable about a rotational axis, a drive unit which is arranged outside the reaction space and can be coupled to the agitator shaft and is configured to drive the agitator shaft in a first rotational direction and a second rotational direction.

The first rotational direction and the second rotational direction may in particular be opposite one another. In particular, the first rotational direction may define a clockwise rotation of the agitator shaft. Preferably, accordingly the second rotational direction may define a counterclockwise rotation of the agitator shaft.

Preferably, the header plate may have an inside facing the reaction space and an outside facing away from the reaction space with multiple connections.

Particularly preferably, the bioreactor may be a single-use bioreactor.

According to a further aspect, a method is provided for producing an agitator unit, in particular as described herein, of an agitator, and/or an agitator in particular as described herein, comprising the steps: provision of an agitator blade receiving portion, provision of at least one agitator blade, arrangement of the at least one agitator blade on the agitator blade receiving portion so that the at least one agitator blade is movable between a first angular position and a second angular position, such that the at least one agitator blade can be oriented at a first angle of attack in the first angular position and at a second angle of attack in the second angular position.

The agitator blade may preferably be arranged on, in particular in the sense of being fastened or formed on, the agitator blade receiving portion.

In particular, the method may comprise provision of an agitator shaft with an agitator blade receiving portion. It may furthermore be provided that the agitator blade receiving portion is arranged on the agitator shaft.

Preferably, a torsionally stiff rotation element may be arranged on the agitator blade receiving portion, in particular so that the rotation element is mounted so as to be rotatable between the first rotational position and second rotational position, such that the at least one agitator blade can be oriented at a first angle of attack in the first rotational position and at a second angle of attack in the second rotational position.

According to a further aspect, a method is provided for producing a bioreactor, in particular as described herein, comprising the steps: provision of a container, provision of a header plate, provision of an agitator as described herein, arrangement of the agitator on the header plate, placing of the header plate on the container, and connection of the header plate to the container so as to enclose a reaction space.

The method may preferably comprise production of an agitator and/or an agitator unit, in particular as described herein.

A further step of the method may for example be provision of a drive unit. The drive unit may preferably be arranged on the header plate and coupled to the agitator shaft.

According to a further aspect, a method is provided for operating a bioreactor in particular as described herein, comprising an agitator in particular as described herein, and/or an agitator unit in particular as described herein, comprising the steps: setting of a first drive parameter as required in order to drive an agitator shaft in a first rotational direction, so that at least one agitator blade is moved into a first angular position in which the at least one agitator blade is oriented at a first angle of attack, and/or setting of a second drive parameter as required in order to drive the agitator shaft in a second rotational direction, so that the at least one agitator blade is moved into a second angular position in which the at least one agitator blade is oriented at a second angle of attack, wherein the first drive parameter and the second drive parameter can be set alternately.

Preferably, the first rotational direction may describe a clockwise rotation. Preferably, the second rotational direction may describe a counterclockwise direction.

Particularly preferably, one aspect may for example also comprise a use of the agitator described herein and/or the agitator unit described herein for setting as required a first drive parameter for driving an agitator shaft in a first rotational direction, so that at least one agitator blade is moved into a first angular position in which the at least one agitator blade is oriented at a first angle of attack, and/or for setting as required a second drive parameter for driving the agitator shaft in a second rotational direction, so that the at least one agitator blade is moved into a second angular position in which the at least one agitator blade is oriented at a second angle of attack, wherein the first drive parameter and the second drive parameter can be set alternately.

For the advantages, embodiment variants and design details of these further aspects and their possible refinements, reference is made to the preceding description of corresponding features.

### BRIEF DESCRIPTION OF THE DRAWINGS

Exemplary embodiments are described for example with reference to the appended Figures. In the drawings:

FIG. 1 shows exemplary illustrations of an agitator unit having an agitation blade in a first angular position (A) and in a second angular position (B);

FIG. 2 shows an exemplary illustration of an agitator;

FIG. 3 shows an exemplary exploded illustration of an agitator unit;

FIG. 4 shows an exemplary sectional illustration of a rotor blade receiving portion;

FIG. 5 shows an exemplary sectional illustration of an agitator unit;

FIG. 6 shows an exemplary illustration of a bioreactor;

FIG. 7 shows an exemplary method for producing an agitator unit;

FIG. 8 shows an exemplary method for producing a bioreactor; and

FIG. 9 shows an exemplary method for operating a bioreactor.

### DETAILED DESCRIPTION OF THE EMBODIMENTS

Similar elements or those with substantially equivalent function are designated with the same reference signs in the Figures.

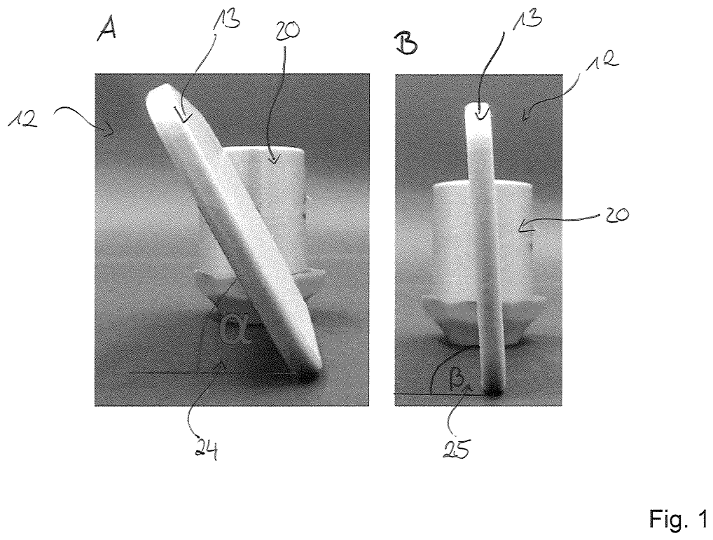

A and B of FIG. 1 each show an exemplary illustration of an agitator unit 12 with an agitator blade 13. The agitator unit 12 also has an agitator blade receiving portion 20 on which the agitator blade 13 is arranged. It should be understood in particular that A and B of FIG. 1 show the same agitator unit 12. The agitator blade 13 is movable between a first angular position shown in A of FIG. 1 and a second angular position shown in B of FIG. 1. The agitator blade 12 may be fastened or formed on the agitator blade receiving portion in particular so as to be movable between the first angular position and the second angular position.

A of FIG. 1 shows the agitator blade 13 at a first angle of attack 24. The first angle of attack 24 is selected such that the agitator blade 13 is arranged obliquely and can hence provide the agitation properties of a sloping blade agitator. The first angle of attack 24 is here less than 90° to a virtual plane arranged orthogonally to a rotational axis of the agitator shaft.

B of FIG. 1 shows the agitator blade 13 at a second angle of attack 25. The second angle of attack 25 is selected such that the agitator blade 13 is arranged substantially vertically and can hence provide the agitation properties of a disc agitator. The second angle of attack 25 is here approximately 90° to a virtual plane arranged orthogonally to a rotational axis of the agitator shaft.

An agitator 10 which, in addition to the agitator unit 12 shown in FIG. 1, comprises in particular also an agitator shaft (not shown in FIG. 1) on which the agitator unit 12 can be fastened or formed, may in particular be used in a bioreactor. Here, preferably, the agitator shaft may be coupled to a drive unit of the bioreactor in order to be able to drive the agitator shaft in particular in a first rotational direction and/or in a second rotational direction. The first rotational direction and the second rotational direction may in particular be opposite one another, preferably clockwise and counterclockwise. Rotation of the agitator shaft in operation, in particular in the presence of a medium in a container of the bioreactor, causes a force to act on the agitator blade 13 opposite the respective rotational direction.

The agitator blade 13 is here arranged in particular movably on the agitator blade receiving portion 20 such that when a force acting against a first rotational direction of the agitator shaft 11 acts on the agitator blade 13, the at least one agitator blade 13 is moved into the first angular position. The agitator blade 13 can also in particular be fixed in the first angular position without fastening means by rotation of the agitator shaft.

In particular, the agitator blade 13 is arranged movably on the agitator blade receiving portion 20 such that when a force acting against the second rotational direction of the agitator shaft 11 acts on the at least one agitator blade 13, the at least one agitator blade 13 is moved into the second angular position. The agitator blade 13 can also in particular be fixed in the second angular position without fastening means by rotation of the agitator shaft.

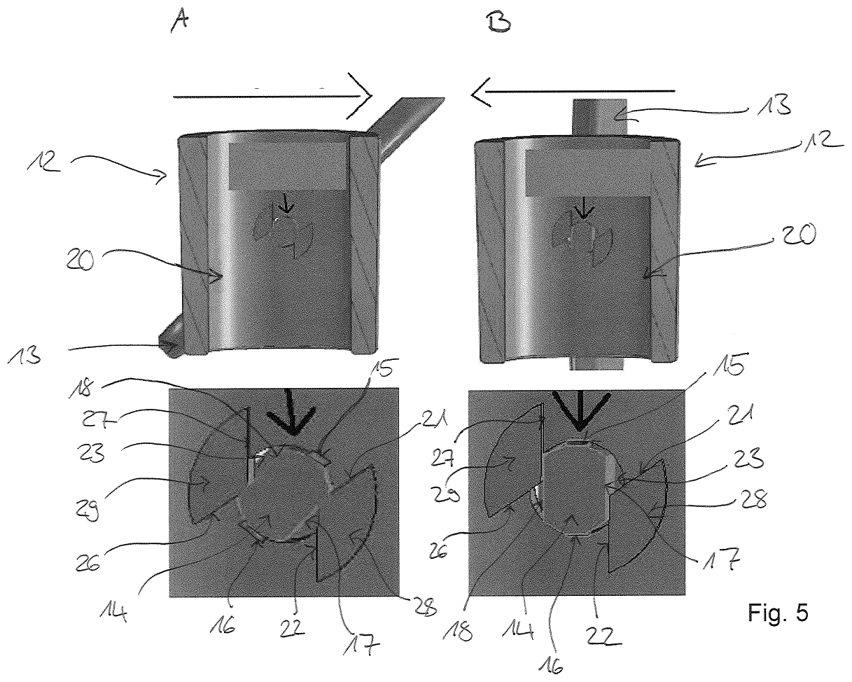

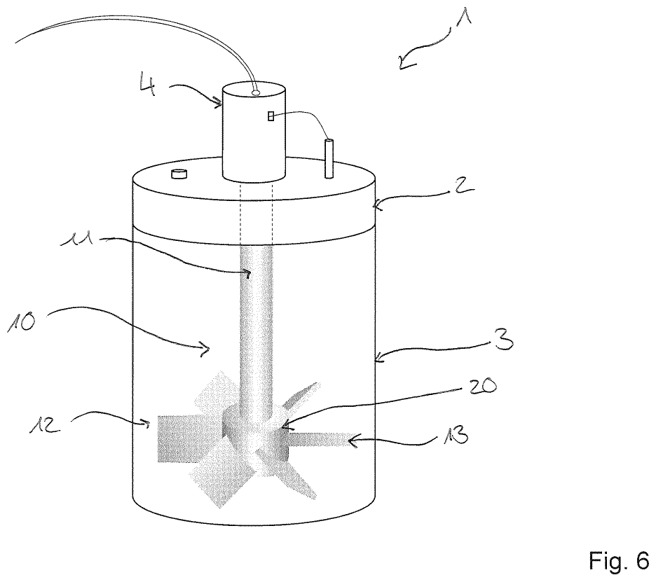

As shown in A and B of FIG. 5 and the further Figures show an exemplary agitator unit 12 with a single agitator blade 13 in order to emphasize the structural and functional features of the agitator. In particular, the design of the agitator unit is not however restricted to the example shown. Rather, it may be understood that the agitator unit 12 has a total of two, three or more agitator blades 13. Particularly preferably, in total six agitator blades 13 are provided, as shown in FIG. 6. It is preferred that all agitator blades 13 of the agitator unit 12 are arranged on the agitator blade receiving portion such that they can be moved between the first angular position and the second angular position.

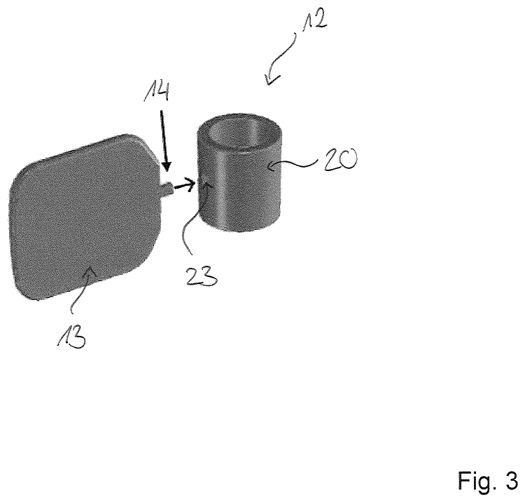

The movement of the agitator blade 13 between the first and second angular positions may in particular be limited and/or guided in the respective direction in order to allow the setting of the corresponding angle of attack, in particular depending on the rotational direction of the agitator shaft. For this, for example, a stop system be provided as described in FIGS. 3 to 5. Alternatively or additionally, a guidance system may be provided.

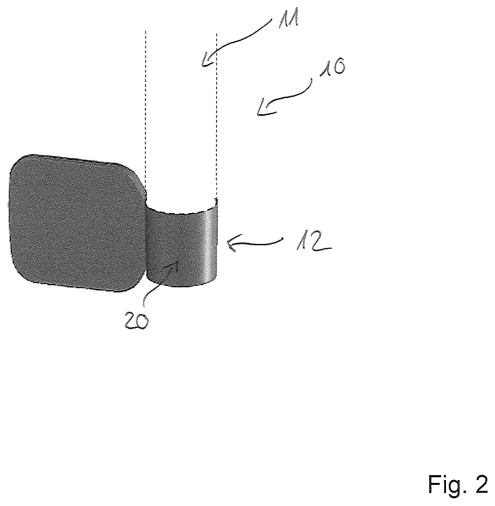

FIG. 2 shows as an example an extract of an agitator 10 with an agitator shaft 11 and an agitator unit 12 which may in particular be configured as described above. The agitator blade receiving portion 20 of the agitator unit 12 may for example be part of the agitator shaft 11. Alternatively, the agitator unit 12 may be an additional element, in particular separable from the agitator shaft 11. The agitator blade receiving portion 20 may here be fastened to the agitator shaft 11. For example, the agitator blade receiving portion 20 may be configured as a ring or a solid cylinder or hollow cylinder in order to be fastened to the agitator shaft 11. For example, the agitator blade receiving portion 20 may be arranged at an end of the agitator shaft 11 facing away from the drive unit. Particularly preferably, snap hooks may be provided for example which allow latching to the agitator shaft 11.

FIG. 3 shows an exploded illustration of an exemplary embodiment of an agitator unit 12. The agitator unit 12 here comprises a rotor blade receiving portion 20 which is formed as a hollow cylindrical middle piece.

The agitator blade 13 comprises a blade region and a torsionally stiff rotation element 14. The rotation element 14 is configured to be received in a receiving opening 23 of the rotor blade receiving portion 20. In particular, the rotation element 14 may be mounted rotatably in the receiving opening 23. This Figure does not show in particular fastening means such as, e.g., snap hooks with which the agitator unit 12 or rotor blade receiving portion 20 can be fastened to an agitator shaft (not shown).

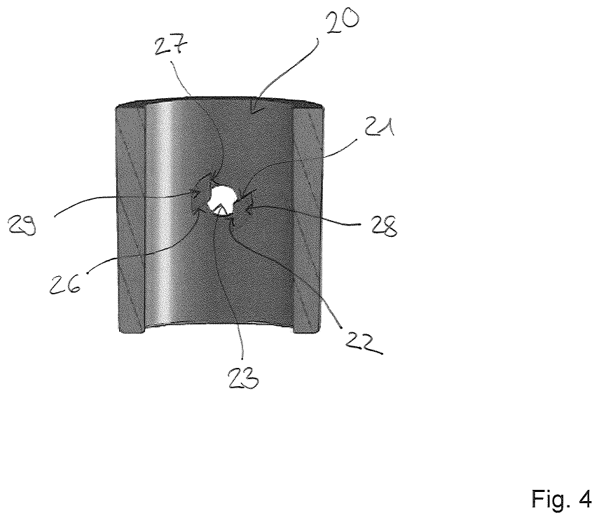

FIG. 4 and A and B of FIG. 5 show a sectional illustration of an exemplary embodiment of a rotor blade receiving portion 20. Insofar as this rotor blade receiving portion 20 is part of an agitator shaft, it may be understood in particular that FIG. 3 shows a sectional illustration of a portion of an agitator shaft. Here, the rotor blade receiving portion 20 is hollow cylindrical and has a receiving opening 23 in which a rotation element can be received.

In the example shown here, a first delimiting element 28 and second delimiting element 29 are arranged on an inner periphery of the rotor blade receiving portion 20. The first delimiting element 28 and the second delimiting element 29 protrude into a region of the receiving opening 23, thus delimiting it. The first delimiting element 28 here has a first stop 21 and a second stop 22, which in the example shown here are formed by stop surfaces of the first delimiting element 28. The second delimiting element 29 has a third stop 26 and a fourth stop 27, which in the example shown here are formed by stop surfaces of the second delimiting element 29.

The first stop 21 and third stop 26 may restrict a rotational movement of a rotation element 14, received in the receiving opening 23, in a first rotational movement. Accordingly, the second stop 22 and fourth stop 27 may restrict a rotational movement of the rotation element 14, received in the receiving opening 23, in a second rotational movement opposite first rotational movement.

The delimiting elements 28, 29 shown in FIG. 4 and A and B of FIG. 5, may provide a stop system. Such a stop system may in particular allow orientation of the agitator blades at a specific angle of attack. In addition, they allow the fixing of the agitator blades at the corresponding angle of attack without fastening means. This provides a particularly simple design of the agitator unit.

For example, according to an embodiment not shown, only the first delimiting element 28 or the second delimiting element 29 may be provided in order to limit the corresponding movement of the rotation element in the rotational directions.

A and B of FIG. 5 each show an exemplary illustration of an agitator unit 12 with an agitator blade 13. It is understood that A and B of FIG. 5 show the same agitator unit 12. A and B of FIG. 5 also each show a detail view of the agitator unit marked by the arrow. The agitator blade 13 is movable between a first angular position, shown in A of FIG. 5, and a second angular position, shown in B of FIG. 5. To allow this movement, the agitator blade 13 has a rotation element 14 which is rotatably mounted in the receiving opening 23. To fasten the rotation element 14 in the receiving opening 23, the rotation element has a first latching element 15 and a second latching element 16 which are latched in the receiving opening 23. In the example shown here, the latching elements 15, 16 are formed at an end of the rotation element 14 facing away from a blade region of the agitator blade 13, and brought into engagement with an edge of the receiving opening 23.

The rotation element 14 has a cross-sectional shape with a first counter-stop 17 and a second counter-stop 18 which, depending on the corresponding rotational position, can bear against the stops 21, 22, 26, 27.

In A of FIG. 5, depending on rotational direction of the agitator shaft and the resulting flow direction of the medium, a force acts on the agitator blade 13 as marked with an arrow in the Figure. This moves the agitator blade into the first angular position and fixes it in this first angular position by means of a stop system without fastening means.

As shown in A of FIG. 5, in the first rotational position, the first counter-stop 17 bears against the first stop 21. In this first rotational position, the second counter-stop 18 also bears against the third stop 26.

In the second rotational position, shown in B of FIG. 5, the first counter-stop 17 bears against the second stop 22, and the second counter-stop 18 bears against the fourth stop 27.

FIG. 6 shows an exemplary bioreactor 1 with a container 3 and a header plate 2. The header plate 2 in the example shown closes the container 3 so as to enclose a reaction space. An agitator 10 is arranged in the reaction space and comprises an agitator shaft 11 with an agitator unit 12. The agitator unit 12 has an agitator blade receiving portion 20 with six agitator blades 13 arranged thereon. The agitator blades 13 in the example shown here are oriented at a first angle of attack so that the agitator unit 12 can fulfil the function of a sloping blade agitator. The agitator blades 12 in the example shown here are movable between a first angular position and a second angular position, so that the agitator blades can be oriented at the first angle of attack or at the second angle of attack, in particular depending on the rotational direction of the agitator shaft 11. By rotation of the agitator shaft 11, the agitator blades 12 can also be fixed in the respective angular position, in particular without fastening means.

Outside the reaction space, a drive unit 4 is also arranged on the coupling plate 2. The drive unit 4 is coupled to the agitator shaft 11. Thus the agitator shaft 11 can be driven either in a first rotational direction or in a second rotational direction. Thanks to this embodiment, the agitation properties can in particular be modified depending on the rotational direction, in particular within a continuous process.

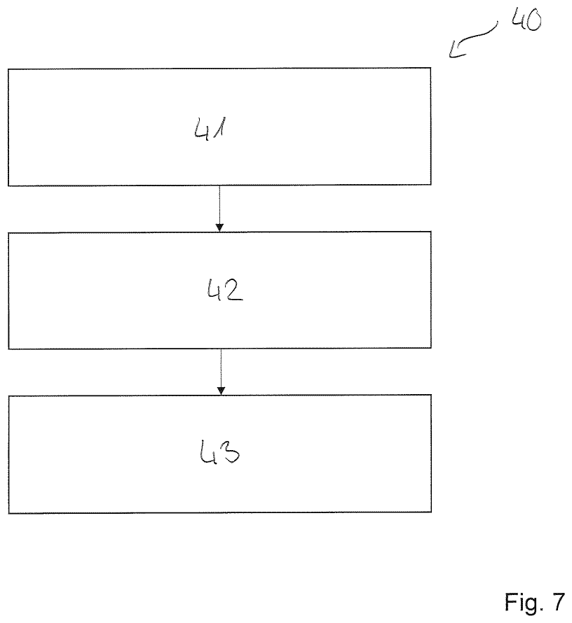

FIG. 7 shows the method steps of a method 40 for producing an agitator unit, as described herein, of an agitator, and/or an agitator, as an example. The method comprises the steps of provision 41 of an agitator blade receiving portion, provision 42 of at least one agitator blade and arrangement 43 of the at least one agitator blade on the agitator blade receiving portion so that the at least one agitator blade is movable between a first angular position and the second angular position, such that the at least one agitator blade can be oriented at a first angle of attack in the first angular position and at a second angle of attack in the second angular position. For example, the method may comprise additional steps for producing the agitator in particular. For example, the method may furthermore comprise the step of provision of an agitator shaft with an agitator blade receiving portion.

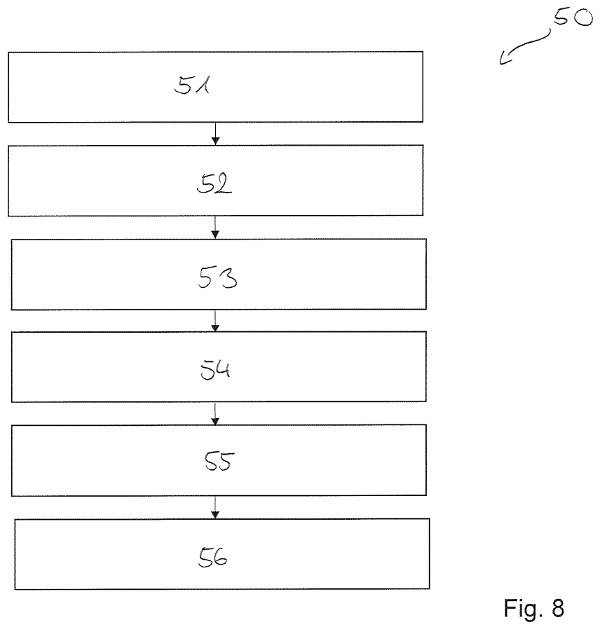

FIG. 8 shows as an example method steps of a method for producing a bioreactor. The method provides the provision 51 of a container and the provision 52 of a header plate. In step 53, an agitator as described herein is provided and in step 54 arranged on the header plate. Finally, in step 55 the method provides the placing of the header plate on the container, and in step 56 finally the connection of the header plate to the container so as to enclose a reaction space.

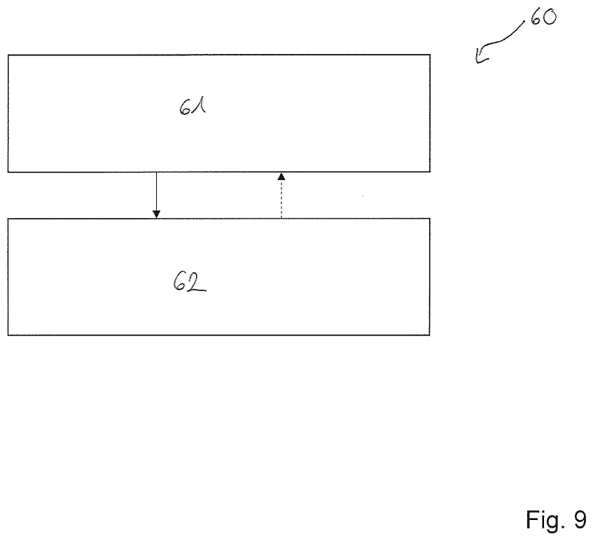

FIG. 9 shows as an example method steps of a method for operating a bioreactor. In step 61, depending on requirement, i.e., in particular depending on the necessary agitation properties and/or properties of the medium, a first drive parameter is set. Thus the agitator shaft is driven in a first rotational direction so that the at least one rotor blade of the agitator unit of the agitator is moved into the first angular position in which the at least one agitator blade is oriented at the first angle of attack. When required, i.e. in particular depending on necessary agitation properties and/or properties of the medium, in step 62 a second drive parameter may be set for driving the agitator shaft in a second rotational direction. Thus the at least one agitator blade can be moved into a second angular position in which the at least one agitator blade is oriented at the second angle of attack. Step 61 and 62 may be set alternately in particular within a process. Thus the agitation properties can be adapted during the process.

1.-13. (canceled)

14. An agitator for a bioreactor, comprising:
an agitator shaft having an agitator unit with an agitator blade receiving portion and at least one agitator blade arranged on the agitator blade receiving portion and movable between a first angular position and a second angular position;
wherein the at least one agitator blade is oriented at a first angle of attack in the first angular position and at a second angle of attack in the second angular position, and wherein the at least one agitator blade comprises a torsionally stiff rotation element mounted on the agitator blade receiving portion so as to be rotatable between a first rotational position and a second rotational position;
wherein the at least one agitator blade is oriented at the first angle of attack in the first rotational position and at the second angle of attack in the second rotational position;
wherein the agitator blade receiving portion comprises at least one receiving opening in which the rotation element is mounted to be rotatable between the first rotational position and the second rotational position; and
wherein the agitator unit further comprises:
a first stop which in the first angular position limits a movement of the at least one agitator blade in a first movement direction and a second stop which in the second angular position limits a movement of the at least one agitator blade in a second movement direction, wherein the first stop and/or the second stop is arranged on or formed by the agitator blade receiving portion, and wherein the receiving opening forms the first stop and/or the second stop or is delimited by the first stop and/or the second stop.

15. The agitator as claimed in claim 14, wherein:
the at least one agitator blade is movably arranged on the agitator blade receiving portion such that when a force acting against a first rotational direction of the agitator shaft acts on the at least one agitator blade, the at least one agitator blade is moved into the first angular position and/or fixed in the first angular position without fastening means; and/or
the at least one agitator blade is movably arranged on the agitator blade receiving portion such that when a force acting against a second rotational direction of the agitator shaft acts on the at least one agitator blade, the at least one agitator blade is moved into the second angular position and/or fixed in the second angular position without fastening means.

16. The agitator as claimed in claim 14, wherein the rotation element is fixed by a form fit in the receiving opening.

17. The agitator as claimed in claim 16, wherein the rotation element is latched in the receiving opening.

18. The agitator as claimed in claim 14, further comprising a guide device configured to guide a movement of the agitator blade between the first angular position and the second angular position.

19. The agitator as claimed in claim 14, wherein the agitator blade receiving portion and the agitator shaft are formed as one piece.

20. The agitator as claimed in claim 14, wherein a middle piece arranged on the agitator shaft forms the agitator blade receiving portion.

21. The agitator as claimed in claim 20, wherein the middle piece is fastened to the agitator shaft.

22. The agitator as claimed in claim 20, wherein the middle piece is formed as a solid cylinder or a hollow cylinder or a ring.

23. The agitator as claimed in claim 14, wherein:
the first angle of attack is less than 90° or equal to 90° to a virtual plane arranged orthogonally to a rotational axis of the agitator shaft; and/or
the second angle of attack is more than 90° or equal to 90° and less than 180° to the virtual plane arranged orthogonally to a rotational axis of the agitator shaft.

24. The agitator as claimed in claim 14, wherein:
the agitator shaft and/or the agitator unit are formed at least partly from or comprise plastic or are formed from or comprise special steel.

25. The agitator as claimed in claim 24, wherein:
the agitator shaft and/or the agitator unit are produced at least partially in an injection molding process or in an additive manufacturing process.

26. The agitator as claimed in claim 14, comprising:
at least one disc agitator arranged on the agitator shaft axially spaced from the agitator unit;
at least one sloping blade agitator arranged on the agitator shaft axially spaced from the agitator unit; and
at least one further agitator unit arranged on the agitator shaft axially spaced from the agitator unit and having a further agitator blade receiving portion and at least one further agitator blade arranged on the further agitator blade receiving portion and movable between two different angular positions in which the at least one further agitator blade is oriented at different angles of attack.

27. An agitator unit for equipping an agitator shaft, the agitator unit comprising:
an agitator blade receiving portion; and
at least one agitator blade arranged on the agitator blade receiving portion and movable between a first angular position and a second angular position;
wherein the at least one agitator blade is oriented at a first angle of attack in the first angular position and at a second angle of attack in the second angular position, wherein the at least one agitator blade comprises a torsionally stiff rotation element mounted on the agitator blade receiving portion to be rotatable between a first rotational position and a second rotational position;
wherein the at least one agitator blade is oriented at the first angle of attack in the first rotational position and at the second angle of attack in the second rotational position; and
whereon the agitator blade receiving portion comprises at least one receiving opening in which the rotation element is mounted to be rotatable between the first rotational position and the second rotational position;
wherein the agitator unit further comprises:
a first stop which in the first angular position limits a movement of the at least one agitator blade in a first movement direction; and/or
a second stop which in the second angular position limits a movement of the at least one agitator blade in a second movement direction, wherein the first stop and/or the second stop are arranged on or formed by the agitator blade receiving portion and wherein the receiving opening forms the first stop and/or the second stop or is delimited by the first stop and/or the second stop.

28. A bioreactor, comprising:
a header plate and a container, wherein the header plate and the container enclose a reaction space;
an agitator as claimed in claim 14 arranged in the reaction space, wherein the agitator shaft is mounted in a bearing to be rotatable about a rotational axis; and
a drive unit arranged outside the reaction space adapted to be coupled to the agitator shaft and configured to drive the agitator shaft in a first rotational direction and a second rotational direction.

29. A method for producing an agitator unit as claimed in claim 27, comprising the steps of:
providing the agitator blade receiving portion;
providing the at least one agitator blade; and
arranging the at least one agitator blade on the agitator blade receiving portion so that the at least one agitator blade is movable between the first angular position and the second angular position, such that the at least one agitator blade can be oriented at the first angle of attack in the first angular position and at the second angle of attack in the second angular position.

30. A method for producing a bioreactor as claimed in claim 28, comprising the steps of:
providing the container;
providing the header plate;
providing the agitator;
arranging agitator on the header plate;
placing the header plate on the container; and
connecting the header plate to the container to enclose the reaction space.

31. A method for operating a bioreactor as claimed in claim 28, comprising the steps of:
setting a first drive parameter as required to drive the agitator shaft in a first rotational direction, so that at least one agitator blade is moved into a first angular position in which the at least one agitator blade is oriented at a first angle of attack; and/or
setting a second drive parameter as required to drive the agitator shaft in a second rotational direction, so that the at least one agitator blade is moved into a second angular position in which the at least one agitator blade is oriented at a second angle of attack;
wherein the first drive parameter and the second drive parameter can be set alternately.

32. The agitator as claimed in claim 14, wherein:
the rotation element comprises a cross-sectional shape forming a first counter-stop adapted to bear again the first stop in the first rotational position and/or a second counter-stop adapted to bear again the second stop in the second rotational position.

33. The agitator as claimed in claim 32, wherein:
the first counter-stop and the second counter-stop are each formed by a flattened portion.
